(19) United States
(12) Patent Application Publication
(10) Pub. No.: US 2019/0039046 A1
(43) Pub. Date: Feb. 7, 2019

(54) ADSORBENT MATERIAL AND PURIFICATION METHOD

(71) Applicant: HITACHI, LTD., Tokyo (JP)

(72) Inventors: Masashi MARUYAMA, Tokyo (JP); Nanae YAMASHITA, Tokyo (JP); Hiroshi YOSHIDA, Tokyo (JP); Keisuke SHIBUYA, Tokyo (JP)

(73) Assignee: HITACHI, LTD., Tokyo (JP)

(21) Appl. No.: 16/077,577

(22) PCT Filed: Jan. 13, 2017
(86) PCT No.: PCT/JP2017/000909

§ 371 Date: Aug. 13, 2018

(30) Foreign Application Priority Data
Feb. 25, 2016 (JP) 2016-033738

(51) Int. Cl. B01J 20/281; B01J 20/32; B01D 15/38; C07K 1/22

(52) U.S. Cl. CPC B01J 20/281; B01J 20/3263; C07K 1/22; B01J 20/3272; B01D 15/3804; B01J 20/3221

(57) ABSTRACT

The present invention has a subject of desorbing a target substance under mild elusion conditions in affinity purification using a low molecular weight ligand. In the present invention, a polar auxiliary group is introduced to a site adjacent to the low molecular weight ligand to perturb a pKa value of a functional group in the low-molecular-weight ligand, thereby ionizing the low molecular weight ligand under mild pH conditions.

### TECHNICAL FIELD

The present invention relates to an adsorbent and a purification method using the same.

### BACKGROUND ART

The affinity purification of a biomolecules such as a protein as a target substance includes steps of adsorbing the target substance to an affinity column, cleaning non-desorbing ingredients and desorbing the target substance from the column by an eluent. In this case, a solution at a severe pH condition such as a strong acid or a strong base is often used as the eluent. This is because it is necessary to weaken the interaction between an affinity ligand and target substance by electrostatic repulsion by changing the ionized state of the affinity ligand or the target substance. However, under a severe pH circumstance such as due to a strong acid or a strong base, biomolecules are often instable to bring about a risk of degrading the purified target substance. Further, there is also a risk of degrading the affinity ligand or the column material.

Then, an affinity column improving the affinity ligand thereby enabling purification of biomolecules under mild pH conditions has been developed (Patent Literature 1). In the Patent Literature 1, protein A is modified by gene combination so as to respond to a mild acid. In this way, when the affinity ligand is a protein, it is relatively easy to adjust pH response by gene recombination.

However, in a case of a low molecular affinity ligand, since a site that interact with the target substance is small, there is less site that can be modified for adjusting the pH response and the degree of freedom for adjusting the responsivity is low (Non-patent Literature 1). While there has been reported that when a low molecular weight affinity ligand and an amine are copolymerized, the local pH at a site adjacent to the low molecular weight affinity ligand is perturbed (Non-patent Literature 2), this is not a generally applicable method.

### CITATION LIST

### Patent Literature

Patent Literature 1: Japanese Unexamined Patent Application Publication 2010-81966

### Non-Patent Literature

Non-patent Literature 1: Journal of Chromatography B, 2000, 740, 1-15

Non-patent Literature 2: Chemical Science. 2012, 3, 1467-1471.

### SUMMARY OF INVENTION

### Technical Problem

In the affinity purification using a low molecular weight ligand, a target substance is desorbed under mild elution conditions.

### Solution to Problem

A polar auxiliary group is introduced to a site adjacent with a low molecular weight ligand to perturb the pKa value of the functional group of the low molecular weight ligand, thereby ionizing the low molecular weight ligand under mild pH conditions.

### Advantageous Effects of Invention

Since no severe elution conditions are used for the elution of the target substance, degradation can be suppressed. Since no severe pH conditions are used for the elution of the target substance, neutralizing step can be simplified.

### BRIEF DESCRIPTION OF DRAWINGS

FIG. 1 illustrates an affinity adsorbent before and after pH change.

FIG. 2 illustrates a purification unit filled with an adsorbent.

FIG. 3 is a table illustrating the result of measuring the affinity of each of specimens to an antibody in view of the retention time upon passing a specimen through an IgG agarose column.

FIG. 4 is a graph illustrating the behavior of each of the specimens upon elution from an IgG agarose column at a linear gradient.

FIG. 5 is a table illustrating the IgG elution feasibility of various low molecular weight ligands, polar auxiliary groups and spacers.

### DESCRIPTION OF EMBODIMENTS

Preferred embodiments of the present invention are to be described below with reference to drawings, etc. The following descriptions illustrate specific example for the contents of the present invention but the present invention is not restricted to such descriptions but can be modified and changed variously by a person skilled in the art within a range of the technical idea disclosed in the present specification. Further, throughout the drawings for explaining the present inventions, those having identical functions carry same reference numerals, for which repetitive description may sometimes be omitted.

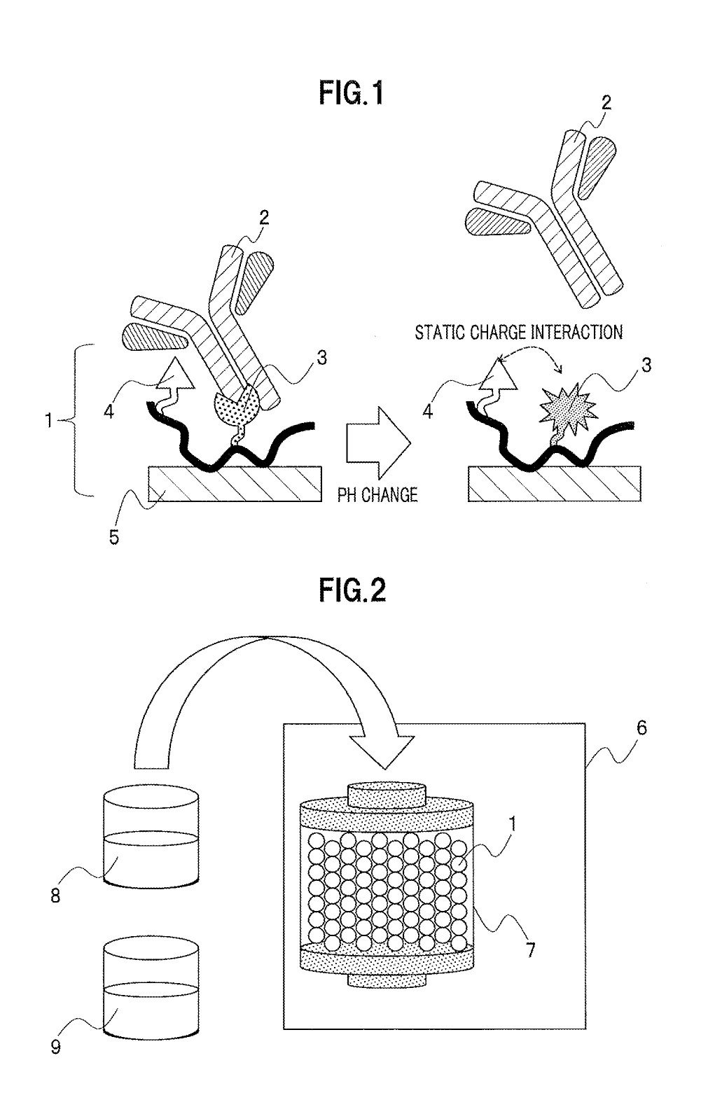

FIG. 1 illustrates an affinity adsorbent 1 before and after pH change. The affinity adsorbent 1 provided in this embodiment comprises a low molecular weight ligand 3 that interacts with a target substance (antibody) 2, a polar auxiliary group 4 to perturb a pKa value of the low molecular weight ligand, and a support 5.

The target substance may be any molecule and any molecule which is instable under severe pH conditions can take an advantageous feature of the present invention.

The low molecular weight ligand 3 contains a functional group that is ionized based on the pH change of a solution. The functional group that is ionized based on the pH change of the solution is a functional group at a pKa of not higher than 17.7 which is higher by 2 than 15.7, that is, a pKa of water and higher than −3.7 which is lower by 2 than −1.7, that is, a pKa of an oxonium ion in water, or a functional group of forming a conjugated acid or a conjugated base at a pKa of 17.7 of higher by 2 than 15.7, that is, a pKa of water and higher than −3.7 which is lower by 2 than −1.7, that is, a pKa of the oxonium ion in water.

The functional group that is ionized based on the pH change of the solution includes, for example, in a case of cationization, a nitrogen-containing hetero ring (pyridyl group, pyrimidyl group, imidazolyl group, oxazolyl group, triazolyl group, triazolyl group, etc.), amino group, ammonium group, imino group, etc. and, in a case of anionization, boronic acid, carboxylic acid, phosphonic acid, phosphinic acid, sulfonic acid, sulfinic acid, alcoholic hydroxyl group, Phenolic hydroxyl group, activated carbonyl compound (malonic acid ester, malonitrile, aceto acetate ester, acetyl acetone, etc.), cyclopentadiene, nitroalkyl group, etc. but they are not restricted to them.

The functional group that is ionized based on the pH change of the solution is neutral before pH change and in a state adsorbable to the target substance 2 in interaction therewith. The functional group that is ionized based on the pH change in the solution is ionized after pH change to desorb the target substance 2 adsorbed so far.

The polar auxiliary group is a functional group in which a functional group that is ionized based on the pH change of the solution containing the low molecular weight ligand is in a state capable of having a charge opposite to the charge in the ionized state of the functional group possesses.

The functional group in which the functional group that is ionized based on the pH change of the solution containing the low molecular weight ligand in a state capable of possessing a charge opposite to the charge in an ionized state includes, for example, a tetraalkyl ammonium in a case of a positive static charge and a fluoro sulfonic acid in a case of a negative static charge, but they are not restricted to them.

Since it is necessary that the low molecular weight ligand and the polar auxiliary group can interact each other, it is preferred that they are spaced by a distance of 30 Å or less.

In the Non-patent Literature 1, a low molecular weight ligand and a secondary amino group are spaced apart by a distance of about 9 Å, but no interaction is recognized due to steric hindrance since the distance is extremely short. In order that the polar auxiliary group provides a sufficient effect, it is preferred that the low molecular weight ligand and the polar auxiliary group are spaced apart by a distance of 10 Å or more.

The low molecular weight ligand may be directly bonded to the support or may be bonded by way of a spacer to the support.

The polar auxiliary group may be bonded directly or may be bonded by way of a spacer to the support.

The low molecular weight ligand and the polar auxiliary group may be introduced on an identical spacer, or they may be introduced on different spacers.

The spacer includes, for example, polyalkylene glycol, poly(N-isopropylacrylamide), poly(N,N-dialkylacrylamide), ε-polylysine, polyamino acid, linear polyethyleneimine, branched polyethyleneimine, polymethacrylate, polyacrylate, polyvinyl ether, etc., but they are not restricted to them.

The support is in a state of plate-shaped, beads-on, fibrous, film-like, or monolithic solid includes, preferably, polysaccharides, synthetic resins or inorganic compounds, or a materials containing composite materials thereof and, more preferably, includes one of agarose, crosslinked agarose, hydrophobic agarose, cellulose, polystyrene, polyalkyl methacrylate, polyglycidyl methacrylate, polyvinyl alcohol, polyvinyl pyrrolidone, polyacryl amide, polysiloxane, polyfluoroethylene, silica, alumina, titania, zirconia, iron oxide, ferrite, hydroxy apatite, and silicate and, more preferably, includes one of agarose, hydrophobic agarose, crosslinked agarose, cellulose, polystyrene, silica, iron oxide, and ferrite but is not restricted to them.

The surface of the support maybe modified with other compounds than the low molecular weight ligand, polar auxiliary group or the spacer.

The affinity adsorbent provided in the present invention can be manufactured, for example, by reacting a spacer having a low molecular weight ligand and a polar auxiliary group, and a column support having an active functional group such as epoxy or NHS ester.

FIG. 2 illustrates a purification unit 6 filled with an adsorbent 1. The purification unit 6 is filled with a great amount of the adsorbent 1 in a portion 7 for filling the adsorbent. When a first solution 8 containing the target substance (antibody) 2 is in contact with the purification unit 6, the target substance 2 is adsorbed to the adsorbent 1. Then, when a second solution 9 at a pH of 4.0 or higher and 10 or lower is in contact with the adsorbent having an antibody adsorbed thereon, the target substance (antibody) 2 can be desorbed from the adsorbent 1.

The pH of the first solution 8 is preferably 4 or higher and 10 or lower, more preferably, 5.0 or higher and 9.0 or lower and, further preferably, 5.5 or higher and 8.5 or lower.

Further, the salt concentration of the second solution 9 is preferably 10 mM or more, more preferably, 20 mM or more and, further preferably, 50 mM or more.

Further, the pH of the second solution 9 is, preferably, 4 or higher and 10 or lower, more preferably, 5.0 or higher and 9.0 or lower and, further preferably, 5.5 or higher and 8.5 or lower.

The second solution 9 which is necessary upon desorption of the target substance from the adsorbent may be any solution so long as pH is different from that of the first solution 8. The pH of the second solution 9 may be higher or lower as compared with the pH of the first solution B, and anyway they may be different from each other. When the pH is equal between them, the target substance is not desorbed from the adsorbent.

When the second solution 9 is in contact with the adsorbent to change pH, the low molecular weight ligand is anionized or cationized. When the low molecular weight ligand is anionized, the low molecular weight ligand is desorbed from the target substance. In this case, when the polar auxiliary group is cationized, anionization of the low molecular weight ligand is facilitated.

When the low molecular weight ligand is cationized, the target substance is desorbed from the low molecular weight ligand. In this case, when the polar auxiliary group is anionized, anionization of the low molecular weight ligand is facilitated.

A liquid to be supplied to the column filled with the affinity adsorbent provided by the present invention is preferably a buffer solution containing water as a solvent but it may also contain other solvents than water.

The feature of the purification process using the column filled with the affinity adsorbent provided by the present invention can be utilized effectively by applying the process, for example, to a target of causing decomposition or formation of coagulation at low pH. Further, since the eluted target substance is obtained in a state dissolved in a buffer solution within a nearly neutral pH range, this can be applied to the succeeding purification process without requiring a neutralization step.

### EXAMPLES

### Example 1

In Example 1, a compound having a low molecular weight ligand and a polar auxiliary group at a distance of 30 Å or less was investigated.

Chloro(amino(hydroxyphenyl)) (amino(hydroxynaphthyl))triazi ne (compound 1) as a low molecular weight ligand was reacted by 0.1 equivalent amount to an amino group of ε-polylysine at 80° C. for three hours in a water-DMF solution. Low molecular weight ingredients were removed by dialysis with purified water and insoluble matters were removed by filtration to obtain a derivative in which (amino(hydroxyphenyl)) (amino(hydroxynaphthyl))triazine was introduced by about 10% to the side chain of ε-polylysine (Sample 1A).

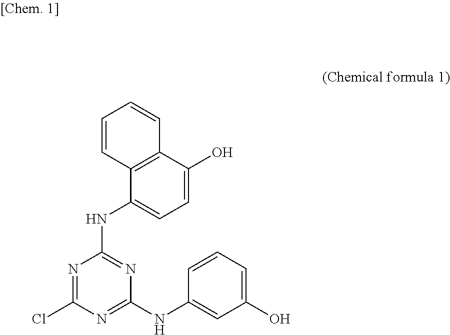 [SMILES: Oc1cccc(Nc2nc(Cl)nc(Nc3ccc(O)c4ccccc34)n2)c1]

To the aqueous solution of Sample 1, pyridyl thioacetic acid as a polar auxiliary group was added by 0.3 equivalent amount and DMT-MM as a condensation agent was added by 0.3 equivalent amount relative to the amino group of the ε-polylysine and reacted at a room temperature for 5 hours. After adding 1 M hydrochloric acid to render the solution acidic, low molecular weight ingredients were removed by dialysis with purified water and insoluble matters were removed by filtration to obtain a derivative in which pyridyl thioacetoamide was introduced by about 20% and (amino(hydroxyphenyl)) (amino(hydroxynaphthyl))triazine was introduced by about 10% to the side chain of ε-polylysine (Sample 2A). As a result of analysis using 1H MNR and molecular modeling, the average distance between the low molecular weight ligand and the polar auxiliary group in the sample 2 was 20 Å.

### Comparative Example 1

In Comparative Example 1, a compound having a low molecular weight ligand and a polar auxiliary group which were at a distance of 30 Å or more was investigated.

Chloro(amino(hydroxyphenyl)) (amino(hydroxynaphthyl))triazine as a low molecular weight ligand was reacted by 0.02 equivalent amount to the amino group of ε-polylysine in a water-DMF solution at 80° C. for three hours. Low molecular weight ingredients were removed by dialysis with purified water and insoluble ingredients were removed by filtration to obtain a derivative in which (amino(hydroxyphenyl)) (amino(hydroxynaphthyl))triazine was introduced by about 4% to the side chain of ε-polylysine (Sample 1B).

A pyridyl thioacetic acid as a polar auxiliary group was added by 0.1 equivalent amount and DMT-MM as a condensation agent was added by 0.1 equivalent amount relative to the amino group in the aqueous solution of the Sample 1 and reacted at a room temperature for 5 hours. After adding 1 M hydrochloric acid to render the solution acidic, low molecular weight ingredients were removed by dialysis with purified water and insoluble matters were removed by filtration to obtain a derivative in which about 6% of pyridyl thioacetamide and about 4% of (amino(hydroxyphenyl)) (amino(hydroxynaphthyl))triazine were introduced to the side chain of ε-polylysine (Sample 2B). As a result of analysis using 1H MNR and amide molecular modeling, the average distance between the low molecular weight ligand and the polar auxiliary group in the Sample 2 was 40 Å.

### Comparative Example 2

In Comparative Example 2, a compound having only the low molecular weight ligand was investigated.

Phenyl thioacetic acid as a polar auxiliary group was added by 0.3 equivalent amount and DMT-MM as a condensation agent was added by 0.3 equivalent amount to the amino group in the aqueous solution of Sample 1 and reacted at a room temperature for 5 hours. After adding 1 M hydrochloric acid to render the solution acidic, low molecular weight ingredients were removed by dialysis with purified water and insoluble ingredients were removed by filtration to obtain a derivative in which about 20% of phenyl thioacetamide and about 10% of (amino(hydroxyphenyl)) (amino(hydroxynaphthyl))triazine were introduced to the side chain of the ε-polylysine (Sample 3).

### Comparative Example 3

In Comparative Example 3, a compound comprising only the polar auxiliary group was investigated

Pyridyl thioacetic acid as a polar auxiliary group was added by 0.3 equivalent amount and DMT-MM as a condensation agent was added by 0.3 equivalent amount relative to the amino group of an aqueous solution of ε-polylysine and reacted at a room temperature for 5 hours. After adding 1 M hydrochloric acid to render the solution acidic, low molecular weight ingredients were removed by dialysis with purified water and insoluble ingredients were removed by filtration to obtain a derivative in which pyridyl thioacetamide was introduced by about 20% to the side chain of the ε-polylysine (Sample 4).

### Comparative Example 4

In Comparative Example 4, ε-polylysine as a spacer was investigated (Sample 5).

### >Affinity Measurement<

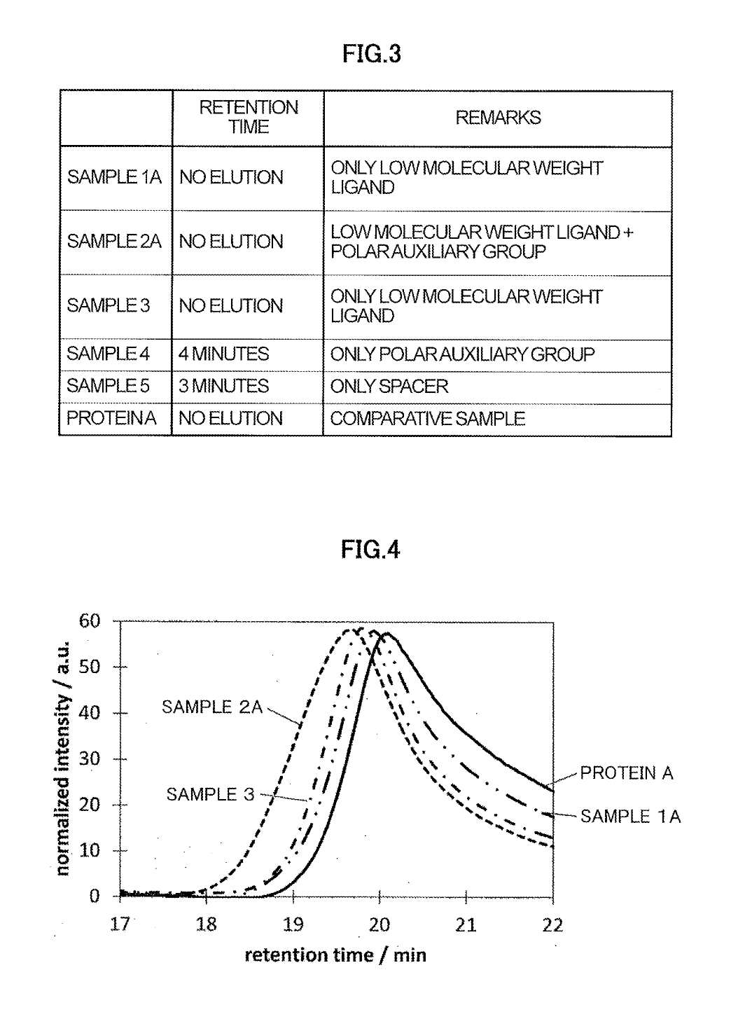

An antibody affinity of each of the Samples 1A, 2A, 3 to 5, and protein A was measured in view of the retention time upon passing the sample through an IgG agarose column (FIG. 3). Samples 1A, 2A, and 3 to 5 were prepared respectively as PBS solutions (pH 7.4, 5 mM) and liquid was passed at a flow rate of 0.5 mL/min to an IgG agarose column of 5 mm diameter, and they were washed with 10 CV of PBS (pH 7.4, 5 mM) (FIG. 3).

As a result, Samples 1A, 2A, and 3 were adsorbed to the IgG agarose column and were not eluted. Then, it was found that the polar auxiliary group does not lower the affinity of the low molecular weight ligand to IgG.

Further, Samples 4 and 5 were eluted by cleaning.

### <Investigation on Antibody Elution>

For IgG columns having the samples 1 to 3 and protein A adsorbed thereon, the columns were cleaned while replacing solvents of composition at a linear gradient from PBS (pH 7.4, 5 mM) 100% to PBS (pH 7.4, 5 mM) 20% and acetic acid buffer solution (pH 4.0 containing NaCl at 0.01 mM and 0.5 mM) and the elution time was measured (FIG. 4).

As a result, the Sample 2A containing the pyridyl group as the polar auxiliary group eluted first of all and the Sample 1A and the Sample 3 not having the polar auxiliary group eluted late Further, the Sample 1B and the Sample 2B showed no change in the elution time. This revealed that when the polar auxiliary group is at the site adjacent to the low molecular weight ligand, this provides an effect of easy elution.

### Example 2

In Example 2, a sample having a low molecular weight ligand and a polar auxiliary group was immobilized to beads and antibody purification was performed.

The Sample 2A was dissolved in a sodium carbonate/sodium hydrogen carbonate buffer solution at pH 10 and reacted with agarose beads having epoxy groups at 40° C. for 12 hours. The obtained agarose beads modified with the Sample 2A were cleaned with PBS (pH 7.4, 5 mM) and they were filled in an empty column (column A).

After passing a solution containing IgG through the column A and cleaning the column by 5 CV of PBS (pH 7.4, 5 mM). When PBS (pH 5, 8.50 mM) was passed, IgG could be purified at a recovery rate of 85%.

### Comparative Example 5

In Comparative Example 5, a sample having a low molecular weight ligand and not having a polar auxiliary group was immobilized to beads and antibody purification was performed.

The Sample 3 was dissolved in a sodium carbonate/sodium hydrogen carbonate buffer solution at pH 10 and reacted with agarose beads having epoxy groups at 40° C. for 12 hours. The obtained agarose beads modified with the Sample 3 were cleaned with PBS (pH 7.4, 5 mM) and filled in an empty column (column B).

After passing a solution containing IgG through the column B and cleaning the column with 5 CV of PBS (pH 7.4, 5 mM), when PBS (pH 5.8, 50 mM) was passed, the IgG recovery rate was 15%, which was lower compared with the system having the polar auxiliary group (column A).

### Comparative Example 6

In Comparative Example 6, a sample having a low molecular weight ligand and a polar auxiliary group were immobilized to the beads so that they were spaced apart by a distance of 30 Å or more and antibody purification was performed.

A mixture of the Sample 3 and the Sample 4 was dissolved in a sodium carbonate/sodium hydrogen carbonate buffer solution at pH 10 and reacted with the agarose beads having epoxy groups at 40° C. for 12 hours. The obtained agarose beads modified with the Sample 3 and the Sample 4 were cleaned with PBS (pH 7.4, 5 mM) and they were filled in an empty column (column C). In view of the immobilized amount of the Sample 3 and the Sample 4 and the specific surface area of the beads, an average distance between the low molecular weight ligand and the polar auxiliary group was 100 Å.

After passing a solution containing IgG through the column B and cleaning the column with PBS (pH 7.4, 5 mM) and when the PBS (pH 5.8, 50 mM) was passed, since the IgG recovery rate was 15% and this was equivalent with that of the column B, it was suggested that the polar auxiliary group should be present at the site adjacent to the low molecular weight ligand to facilitate elution by the polar auxiliary group.

### Example 3

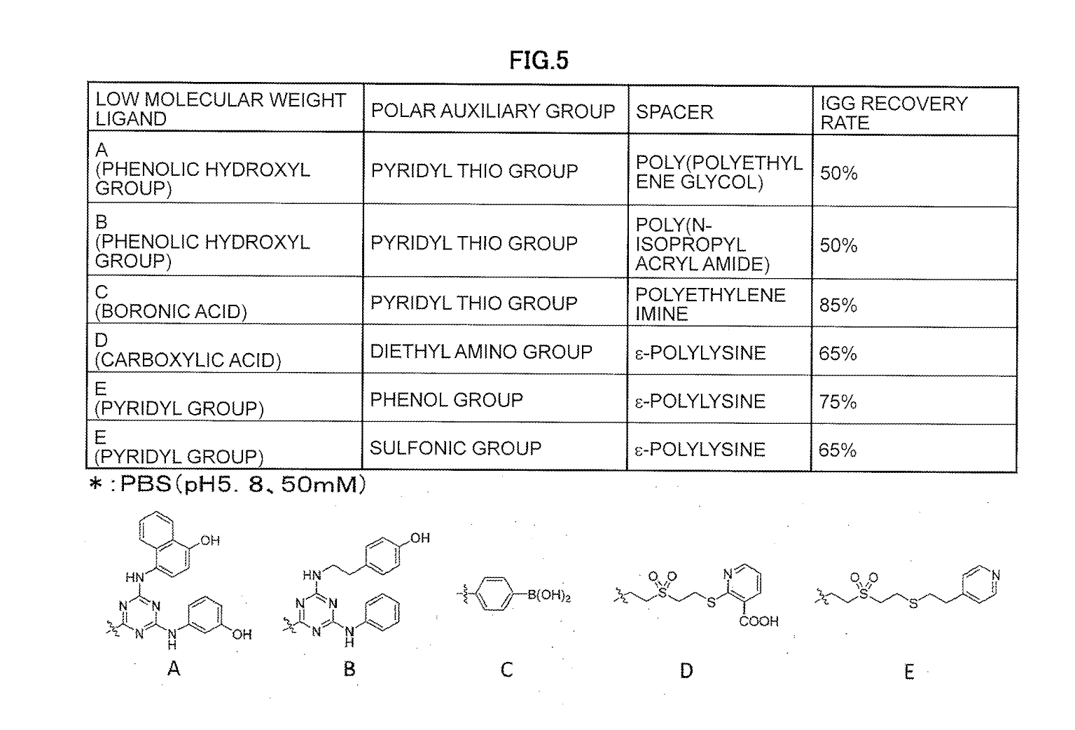

In Example 3, feasibility of IgG elution was experimentally investigated using the column identical with that in Example 2 for the combination of various low molecular weight ligands, the polar auxiliary groups, and the spacers (FIG. 5).

### LIST OF REFERENCE SIGNS

1: adsorbent
2: target substance
3: low molecular weight ligand
4: polar auxiliary group
5: support
6: purification unit
7: sight filled with adsorbent
8: first solution
9: second solution

1. An adsorbent comprising a low molecular weight ligand, a polar auxiliary group, and a support,
wherein the low molecular weight ligand has a functional group which is ionized based on the pH change of a solution,
the polar auxiliary group is in a state capable of having a charge opposite to a charge possessed by the low molecular weight ligand in an ionized state, and
the average distance between the low molecular weight ligand and the polar auxiliary group is 10 Å or more and 30 Å or less.

2. The adsorbent according to claim 1,
wherein the low molecular weight ligand has a functional group that is anionized based on the pH change, and
the polar auxiliary group has a cationic polar auxiliary group.

3. The adsorbent according to claim 2,
wherein the functional group that is anionized based on the pH change includes one of phenolic hydroxyl group, boronic acid, carboxylic acid, phosphoric acid, and sulfonic acid, and
the cationic polar auxiliary group includes one of nitrogen-containing hetero cyclic ring, amino group, and ammonium group.

4. The adsorbent according to claim 1,
wherein the low molecular weight ligand has a functional group that is cationized based on the pH change, and
the polar auxiliary group has an anionic polar auxiliary group.

5. The adsorbent according to claim 4,
wherein the functional group that is cationized based on the pH change includes one of a nitrogen-containing heterocyclic ring, amino group, and ammonium group, and
the anionic polar auxiliary group includes one of phenolic hydroxyl group, boronic acid, carboxylic acid, phosphonic acid, and sulfonic acid.

6. The adsorbent according to claim 1,
wherein the low molecular weight ligand and the polar auxiliary group are bonded by way of a spacer.

7. The adsorbent according to claim 6,
wherein the spacer includes one of polyalkylene glycol, poly(N-isopropyl acryl amide), poly(N,N-dialkyl acryl amide), ε-polylysine, polyamino acid, linear polyethylene imide, branched polyethylene imine, polymethacrylate, a polyacrylate, and a polyvinyl ether.

8. The absorbent according to claim 1,
wherein the support includes one of agarose, crosslinked agarose, hydrophobic agarose, cellulose, polystyrene, silica, iron oxide, and ferrite.

9. The absorbent material according to claim 1,
wherein the low molecular weight ligand is capable of absorbing an antibody.

10. A purification unit wherein the adsorbent according to claim 1 is filled.

11. A method of purifying a target substance, comprising:
a step of bringing a first solution containing a target substance into contact with an adsorbent, thereby adsorbing the target substance to the adsorbent, and
a step of bringing a second solution into contact with the adsorbent to which the target substance has been adsorbed, thereby desorbing the target substance from the adsorbent,
wherein the pH value of the first solution and the pH value of the second solution are different.

12. The purification method according to claim 11,
wherein the adsorbent includes a low molecular weight ligand, a polar auxiliary group, and a support,
the low molecular weight ligand includes a functional group that is ionized based on pH change in the solution, and
the polar auxiliary group is in a state capable of having a charge opposite to a charge possessed by the low molecular weight ligand in an ionized state, and
the average distance between the low molecular weight ligand and the polar auxiliary group is 10 Å or more and 30 Å or less.

13. The purification method according to claim 11,
wherein the pH of the first solution and the pH of the second solution are 4 or higher and 10 or lower.

14. The purification method according to claims 11,
wherein the target substance is an antibody.
